(19) United States
(12) Patent Application Publication
(10) Pub. No.: US 2020/0071834 A1
(43) Pub. Date: Mar. 5, 2020

(54) COATING METHOD OF APATITE USING LASER

(71) Applicant: KOREA INSTITUTE OF SCIENCE AND TECHNOLOGY, Seoul (KR)

(72) Inventors: Hojeong JEON, Seoul (KR); Seung Hoon UM, Seoul (KR); Yongwoo CHUNG, Seoul (KR); Hyunseon SEO, Seoul (KR); Yu Chan KIM, Seoul (KR); Myoung-Ryul OK, Seoul (KR); Hyun Kwang SEOK, Seoul (KR)

(73) Assignee: KOREA INSTITUTE OF SCIENCE AND TECHNOLOGY, Seoul (KR)

(21) Appl. No.: 16/555,079

(22) Filed: Aug. 29, 2019

(30) Foreign Application Priority Data
Sep. 3, 2018 (KR) 10-2018-0104453

(51) Int. Cl. C23C 22/03; A61L 27/06; A61L 27/32; C23C 22/78; C23C 22/82

(52) U.S. Cl. CPC C23C 22/03; A61L 27/06; C23C 22/82; C23C 22/78; A61L 27/32

(57) ABSTRACT

Provided is a method of forming an apatite coating, including brining an apatite-forming precursor solution including Ca²⁺ ions and PO₄³⁻ ions into direct contact with at least one region of the substrate, emitting a laser beam onto the region of the substrate in direct contact with the precursor solution through the precursor solution, and forming apatite on the region onto which the laser beam is emitted.

### CROSS-REFERENCE TO RELATED APPLICATION

This application claims the benefit of Korean Patent Application No. 10-2018-0104453, filed on Sep. 3, 2018, in the Korean Intellectual Property Office, the disclosure of which is incorporated herein in its entirety by reference.

### BACKGROUND

### 1. Field

The present invention relates to a method of forming an apatite coating using a laser, and more particularly, to a method of forming an apatite coating on the surface of a substrate by bringing a precursor solution including Ca²⁺ ions and PO₄³⁻ ions into contact with the substrate and emitting a laser beam thereto.

### 2. Description of the Related Art

Titanium-based alloys that are the most widely used metallic biomaterials for medical purposes are reported as superior materials to conventional biometals due to low modulus of elasticity, excellent biocompatibility, and high corrosion resistance. However, bioinert titanium-based alloys cannot directly induce osteogenesis and require a long treatment time to bond to adjacent bones, and a spontaneously formed titanium oxide coating is too thin to induce regeneration of adjacent bone tissue since the coating rapidly disappears.

Thus, bioactivity is imparted to an implant by surface treatment to solve problems as described above such as direct bonding failure between the implant and the bone and relaxation for reducing a time of implant-bond integration time. The bioactivity of titanium, used as a main material for implants, is further improved by physical or chemical surface treatment, thereby reducing a healing time after an implant is introduced into a human body, and research has been conducted into more effective surface treatment.

In this regard, hydroxyapatite has been used as a material applied to the surface of titanium for the surface treatment. Hydroxyapatite is a basic component constituting hard tissue of the human body and has been used as a bone graft material or a bone regeneration material. Hydroxyapatite with a chemical structure of Ca₁₀(PO₄)₆(OH)₂ is distributed in dental enamel of the human body mainly in the outermost enamel layer having a thickness of 1 to 2 mm. Hydroxyapatite is known to have a remineralization effect of directly filling up micropores of demineralized enamel.

Various methods such as anodizing, sol-gel method, plasma spraying, chemical vapor deposition (CVD), and plasma electrolytic oxidation (PEO) have been used to form a hydroxyapatite coating on the surface of a substrate such as titanium by surface treatment.

First, the anodizing is a method of forming a relatively thick layer of an oxide and a metal salt on the surface of a metal using an external power source. A metal, an oxide layer of which is to be formed, is installed at an anode, and another insoluble metal is brought into contact with a cathode to allow a current to flow in an electrolyte. By flowing a current for anodizing, a thin film of an hydroxide of the metal is formed at a very low voltage, and a metal oxide layer is formed at a voltage of about 10 V. However, once the oxide layer is formed, resistance increases causing concentration of an internal stress in the metal oxide layer, and the oxide layer is destroyed at 70 V. When the voltage is increased again, a second porous oxide layer is formed. During this process, sparks may occur, electrical efficiency may decrease since the oxide layer is formed by forcibly applying electricity thereto, a local area where the sparks occur receives thermal stress to deteriorate physical properties of titanium, and adhesion decreases to deteriorate final physical properties thereof.

The sol-gel method is a method of preparing a solution converted into a gel by hydrolysis or polymerization using alcohol, water, acid, and the like to form a coating film. A homogenized solution is applied to a substrate in a state having a relatively low viscosity and a coating layer is formed on the substrate by gelation. A wet coating method such as dip-coating, which is an application of the sol-gel method, is a low temperature process and has advantages of forming a coating layer regardless of an area and controlling a thickness or microstructure of the coating layer. However, there may be disadvantages of requirement of additional post-heat treatment for crystallization, limited formation of a flat coating, and requirement of an adhesive inserted into an intermediate layer to obtain a sufficient binding force between the coating and the substrate.

The plasma spraying, a thermal spraying method, is a process of depositing a metallic material and a nonmetallic material, such as ceramic, having a high melting point on a substrate in a molten or semi-molten state. Although plasma spraying is advantageous in that the material and the size of the substrate are not limited without causing deformation in the substrate, this method is applicable on the spot, a thick coating may be formed, the thickness of a coating is easily controlled, and various types of coating materials may be used, it is difficult to apply plasma spraying to implants since the structure has a porosity of 0.6 to 15%, a ceramic coating layer formed on titanium by mechanical bonding instead of metallic bonding is weak against impact, and adhesion between the coating layer and the substrate is weak.

The plasma electrolytic oxidation (PEO) is a surface treatment process of forming a dense coating layer with excellent mechanical stability by inducing microdischarge on the surface of a metallic material immersed in an electrolyte. Properties of the coating layer formed by the PEO are controlled by various process parameters including the electrolyte. Particularly, electrolyte conditions and current density are the most important factors affecting formation and physical properties of the coating layer. The electrolyte generally used herein is potassium phosphate, sodium phosphate, glycerol phosphate, and phosphate. Although such additives generally facilitate the plasma electrolytic oxidation process by increasing electrical conductivity and pH, the additives may react with hydroxyapatite to lower purity and form another compound. Thus, there are problems of a low crystallinity of hydroxyapatite on the surface of an implant and a low content in the coating layer.

### SUMMARY

The present invention has been proposed to solve the above problems, and an object of the present invention is to provide a method of forming an apatite coating on a substrate by emitting a laser beam onto the surface of the substrate on which a precursor solution is applied.

However, these problems to be solved are illustrative and the scope of the present invention is not limited thereby.

Additional aspects will be set forth in part in the description which follows and, in part, will be apparent from the description, or may be learned by practice of the presented embodiments.

According to an aspect of the present invention to achieve the object, provided is an apparatus for forming an apatite coating including: a precursor solution container to contain a precursor solution for forming apatite and providing an environment in which the precursor solution is in direct contact with a substrate; and a laser generator disposed to emit a laser beam onto the substrate through the precursor solution contained in the precursor solution container in a state where the precursor solution is in direct contact with the substrate.

According to an embodiment of the present invention, the apparatus may further include a substrate receiving part on which the substrate is placed, wherein the precursor solution container has an opening at one or more positions allowing the precursor solution contained therein to be in direct contact with the substrate.

According to an embodiment of the present invention, the opening of the precursor solution container may have a structure sealed by the substrate.

According to an embodiment of the present invention, the apparatus may further include a substrate receiving part on which the substrate is placed, wherein the substrate receiving part is formed inside the precursor solution container.

According to another aspect of the present invention to solve the problems, provided is a method of forming an apatite coating including: (a) brining an apatite-forming precursor solution including Ca²⁺ ions and PO₄³⁻ ions into direct contact with at least one region of the substrate; (b) emitting a laser beam onto the region of the substrate in direct contact with the precursor solution through the precursor solution; and (c) forming apatite in the region onto which the laser beam is emitted.

According to an embodiment of the present invention, the method may further include (d) partially removing the apatite by removing the precursor solution and emitting a laser beam onto the region on which the apatite is formed after the step (c).

According to an embodiment of the present invention, the precursor solution may be selected from Dulbecco Modified Eagle Medium (DMEM), human blood plasma (HBP), and simulated body fluid (SBF).

According to an embodiment of the present invention, the precursor solution is concentrated to 1 to 400 times for use.

According to an embodiment of the present invention, the emitting of the laser beam may be performed by repeatedly scanning the laser beam once or more times in one direction by a predetermined distance.

According to an embodiment of the present invention, the emitting of the laser beam may be performed by repeatedly scanning the laser beam once or more times in a zigzag direction by a predetermined distance.

According to an embodiment of the present invention, the substrate may include one of titanium (Ti), a titanium alloy, magnesium (Mg), and a magnesium alloy.

### BRIEF DESCRIPTION OF THE DRAWINGS

These and/or other aspects will become apparent and more readily appreciated from the following description of the embodiments, taken in conjunction with the accompanying drawings in which:

FIGS. 1A and 1B show schematic diagrams of apparatuses for forming apatite coatings according to an embodiment;

FIGS. 2A and 2B show a scanning electron microscope (SEM) image of apatite and energy dispersive spectrometry (EDS) results thereof according to an embodiment;

FIG. 3 shows SEM images of apatite formed according to laser emission conditions according to an embodiment;

FIGS. 4A, 4B, 5A, and 5B are SEM images of apatite formed according to emission directions of laser beams according to an embodiment;

FIGS. 6A and 6B show SEM images indicating changes in surface morphology (roughness and pores) of titanium according to the repetition number of laser beam emission according to an embodiment;

FIG. 7 shows X-ray diffraction (XRD) measurement results of apatite according to an embodiment;

FIGS. 8A to 8D show surface component analysis results after a scratching test according to an embodiment; and

FIGS. 9A and 9B show SEM images of apatite formed on a magnesium substrate and EDS results thereof according to an embodiment.

### DETAILED DESCRIPTION

In the following detailed description, reference is made to the accompanying drawings that show, by way of illustration, specific embodiments in which the invention may be practiced. These embodiments are described in sufficient detail to enable those skilled in the art to practice the invention. It is to be understood that the various embodiments of the invention, although different, are not necessarily mutually exclusive. For example, a particular feature, structure, or characteristic described herein, in connection with one embodiment, may be implemented within other embodiments without departing from the spirit and scope of the invention. In addition, it is to be understood that the location or arrangement of individual elements within each disclosed embodiment may be modified without departing from the spirit and scope of the invention. The following detailed description is, therefore, not to be taken in a limiting sense, and the scope of the present invention is defined only by the appended claims, appropriately interpreted, along with the full range of equivalents to which the claims are entitled. In the drawings, like numerals refer to the same or similar functionality throughout the several views and some elements in the drawings may be exaggerated for descriptive convenience.

Hereinafter, embodiments of the present invention will be described in detail with reference to the accompanying drawings so that these embodiments may be readily implemented by those skilled in the art.

An apparatus for forming an apatite coating according to an embodiment of the present invention includes a precursor solution container to contain a precursor solution for forming apatite, and a laser generator configured to generate a laser beam passing through the precursor solution contained in the precursor solution container.

The precursor solution container provides an environment in which a substrate on which apatite is to be formed is in direct contact with the precursor solution in a state where the precursor is contained therein.

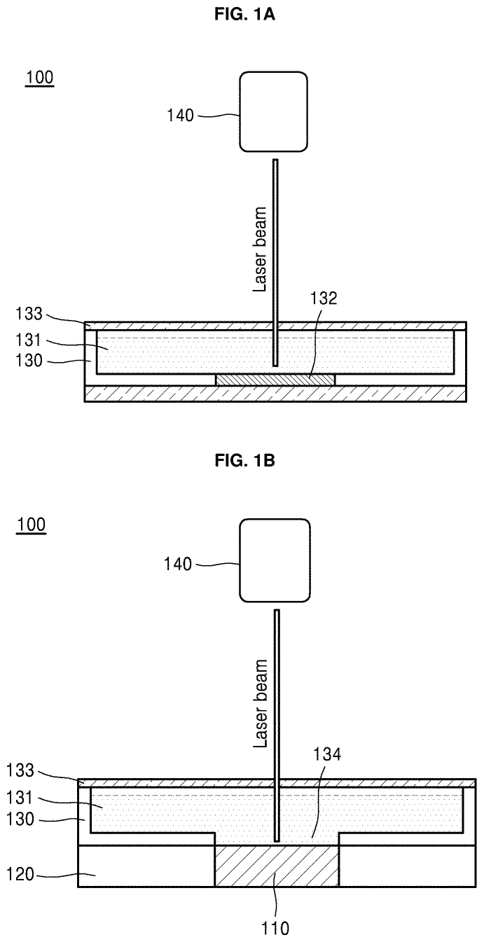

An example of an apatite coating forming apparatus is illustrated in FIG. 1A. Referring to FIG. 1A, an apatite coating forming apparatus 100 includes a vessel-shaped precursor solution container 130 and a laser generator 140 capable of emitting a laser beam from above the precursor solution container 130. The precursor solution container 130 may contain a precursor solution 131. Since the precursor solution container 130 is in the form of a vessel, the substrate 110 may be fixed in the vessel. When the precursor solution 131 is added to the precursor solution container 130 and the substrate 110 is fixed therein, an environment in which the precursor solution 131 is in direct contact with the substrate 110 is formed.

In this regard, a substrate receiving part 132 on which the substrate 110 is placed may be formed at one portion of the precursor solution container 130. Although FIG. 1A illustrates the substrate receiving part 132 in the form of a groove on which the substrate 110 is mounted and fixed, the present invention is not limited thereto and any structure capable of stably accommodate the substrate 110 may be applicable thereto.

Optionally, a portion of the precursor solution container 130 may be open to allow a laser beam to pass therethrough or may be provided with a window 133 formed of a transparent material capable of transmitting the laser beam therethrough.

The substrate 110 may be formed of a material on which an apatite coating is formed, for example, a metal available in living bodies. For example, the substrate 110 may be formed of one of titanium, a titanium alloy, magnesium, a magnesium alloy. In addition, any material required to form an apatite coating, such as a metallic material or a ceramic material, may be used.

The precursor solution 131 is a solution for supplying raw materials for forming apatite and includes Ca²⁺ ions and PO₄³⁻ ions. For example, the precursor solution 131 may be selected from Dulbecco Modified Eagle Medium (DMEM), human blood plasma (HBP), and simulated body fluid (SBF). The precursor solution 131 may be concentrated to increase concentrations of Ca²⁺ ions and PO₄³⁻ ions. Preferably, the precursor solution 131 may be concentrated to 1 to 400 times.

The laser generator 140 is a device configured to emit a laser beam onto a region where the precursor solution 131 is in contact with the substrate 110. When the laser beam with high energy is emitted onto the region where the precursor solution 131 is in contact with the substrate 110, reactions between Ca²⁺ ions and PO₄³⁻ ions are activated in the precursor solution to form an apatite layer on the surface of the substrate 110. In this sense, the laser generator 140 may be a component serving as an energy source for supplying energy for forming apatite.

As the laser generator 140, for example, an ytterbium nanosecond pulsed laser generator or femtosecond pulsed laser generator may be used. In this regard, the nanosecond pulsed laser refers to a laser having a short pulse width of 10⁻⁹ seconds with a pulse time of several nanoseconds, and the femtosecond pulsed laser refers to a laser having a very short pulse width of 10⁻¹⁵ seconds. However, the present invention is not limited thereto, and any laser capable of supplying sufficient energy to the precursor solution to form apatite may also be used.

An apatite coating forming apparatus according to another embodiment of the present invention is illustrated in FIG. 1B.

Referring to FIG. 1B, the apatite coating forming apparatus 100 includes a substrate 110, a substrate receiving part 120, a precursor solution container 130, and a laser generator 140. In the present embodiment, the substrate 110 and the substrate receiving part 120 are located outside the precursor solution container 130. The substrate receiving part 120 supports the substrate 110 to fix the substrate 110 to a predetermined position during laser treatment.

In the present embodiment, the precursor solution container 130 has an opening 134 at one portion such that the precursor solution 131 contained therein may be in direct contact with the substrate 110, and an environment in which the precursor solution 131 is in direction contact with the substrate 110 is formed via the opening 134. A surface of the precursor solution container 130 where the precursor solution 131 contained in the precursor solution container 130 is in direct contact with the substrate 110 constitutes a region to which the laser beam is applied. According to the present embodiment, the precursor solution 131 is locally in direct contact with the substrate 110 through the opening 134.

Optionally, a part of the precursor solution container 130 may be open to allow the laser beam to pass therethrough or may be provided with a window 133 formed of a transparent material capable of transmitting the laser beam therethrough.

Hereinafter, a method of forming an apatite coating on the substrate 110 will be described with reference to the apatite coating forming apparatus 100 illustrated in FIG. 1B.

After the substrate 110 is fixed to a predetermined position using the substrate receiving part 120, the precursor solution container 130 is filled with the precursor solution 131. In this case, the precursor solution 131 needs to be in direct contact with the surface of the substrate 110 through an open surface of the bottom of the precursor solution container 130.

Subsequently, the laser generator 140 emits a laser beam to a region where the precursor solution 131 is in direct contact with the substrate 110 to form an apatite coating on the surface of the substrate 110. In this case, the laser beam generated by the laser generator 140 passes through the precursor solution 131 onto the surface of the substrate 110.

By emitting the laser beam to the precursor solution 131, energy is applied to the precursor solution 131, resulting in generation of apatite on the surface of the substrate 110.

For example, when the Dulbecco Modified Eagle Medium (DMEM) is used as the precursor solution 131, apatite is formed on the surface of the substrate 110 via the reaction of Formula 1 below using the laser beam as an energy source.

6H₃PO₄(aq)+10Ca(OH)₂(aq)→Ca₁₀(PO₄)₆(OH)₂(s)+18H₂O(l) Formula 1

An area, shape, thickness, and the like of the apatite coating formed on the surface of the substrate may be modified by adjusting conditions of the laser beam, e.g., power, frequency, pulse width, scanning method, scan speed, and the like of the laser beam.

For example, in order to form apatite over the entire surface of a substrate, the entire surface of the substrate may be scanned by the laser beam. As another example, in order to locally form apatite on a predetermined region of the substrate, the region of the substrate may be irradiated with or scanned by the laser beam.

As another example, after apatite over the entire area according to the above-described method, an apatite coating having a desired pattern may be formed by removing apatite of a certain area by directly emitting a laser beam to the area without passing through the precursor solution.

Hereinafter, the present invention will be described in more detail with reference to the following examples. However, these examples are made only for illustrative purposes, and the present invention is not be construed as being limited to those examples.

### EXAMPLES

The apatite coating forming apparatus as illustrated in FIG. 1B was fabricated. An titanium alloy of Ti-6Al-4V alloy or magnesium was used as a substrate. A substrate receiving part was manufactured in a mold form using PDMS allowing the substrate to be seated thereon. The substrate was fixed to the PDMS mold. DMEM concentrated to 100 to 400 times was added to the precursor solution container provided on the PDMS mold to which the substrate was fixed. Then, a laser beam was emitted onto the surface of the substrate using a ytterbium nanosecond pulsed fiber laser by scanning to form an apatite coating on the surface of the substrate. A power of the laser beam was selected in the range of 5 to 10 W and a scan speed was selected in the range of 100 to 1000 mm/s. The laser beam was emitted in a method of repeating scanning of the laser beam in one direction by a predetermined distance (one direction method) or a method of repeating scanning of the laser beam in a zigzag direction (zigzag direction method).

The repetition (Mark Loop) was performed from 50 times to 300 times according to conditions for coating formation. Details of the conditions are shown in Table 1 below.

TABLE 1

| | | | Laser | | | |
|---|---|---|---|---|---|---|
| | | Concentration | emission | | Number of | Laser |
| | | ratio of | speed | Power | repetition | emission |
| | Substrate | precursor | (mm/s) | (W) | (Mark loop) | method |
| Example 1 | titanium | 100 times | 500 | 5 | 75 | one |
| | alloy | | | | | direction |
| Example 2 | titanium | 100 times | 500 | 5 | 100 | one |
| | alloy | | | | | direction |
| Example 3 | titanium | 100 times | 500 | 10 | 75 | one |
| | alloy | | | | | direction |
| Example 4 | titanium | 100 times | 500 | 10 | 100 | one |
| | alloy | | | | | direction |
| Example 5 | titanium | 100 times | 1000 | 10 | 50 | one |
| | alloy | | | | | direction |
| Example 6 | titanium | 100 times | 1000 | 10 | 50 | zigzag |
| | alloy | | | | | |
| Example 7 | titanium | 400 times | 500 | 5 | 100 | one |
| | alloy | | | | | direction |
| Example 8 | titanium | 400 times | 500 | 5 | 125 | one |
| | alloy | | | | | direction |
| Example 9 | titanium | 400 times | 500 | 5 | 225 | one |
| | alloy | | | | | direction |
| Example 10 | titanium | 400 times | 500 | 5 | 250 | one |
| | alloy | | | | | direction |
| Example 11 | titanium | 400 times | 500 | 5 | 300 | one |
| | alloy | | | | | direction |
| Example 12 | titanium | 400 times | 500 | 10 | 50 | one |
| | alloy | | | | | direction |
| Example 13 | titanium | 400 times | 500 | 10 | 100 | one |
| | alloy | | | | | direction |
| Example 14 | magnesium | 100 times | 100 | 10 | 50 | one |
| | | | | | | direction |
| Example 15 | magnesium | 100 times | 100 | 18.4 | 50 | one |
| | | | | | | direction |

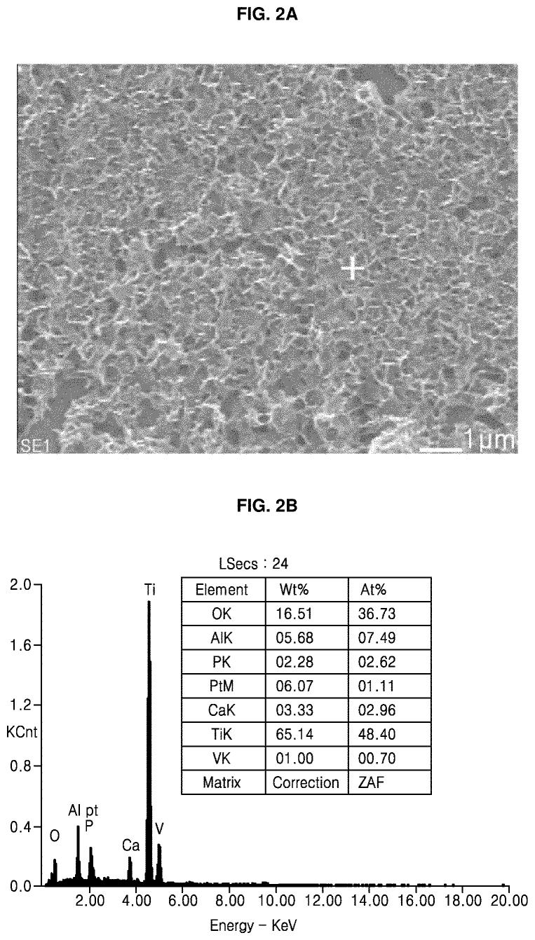

FIGS. 2A and 2B show a scanning electron microscope (SEM) image of apatite formed on the surface of the substrate and composition analysis results thereof by energy dispersive spectrometry (EDS) according to Example 1.

First, referring to FIG. 2A, it was confirmed that a coating having a porous structure was formed on the surface of the titanium alloy used as the substrate. Referring to FIG. 2B, Ca and P peaks were identified as a result of the composition analysis of a product by EDS, indicating that apatite was formed on the surface of the substrate.

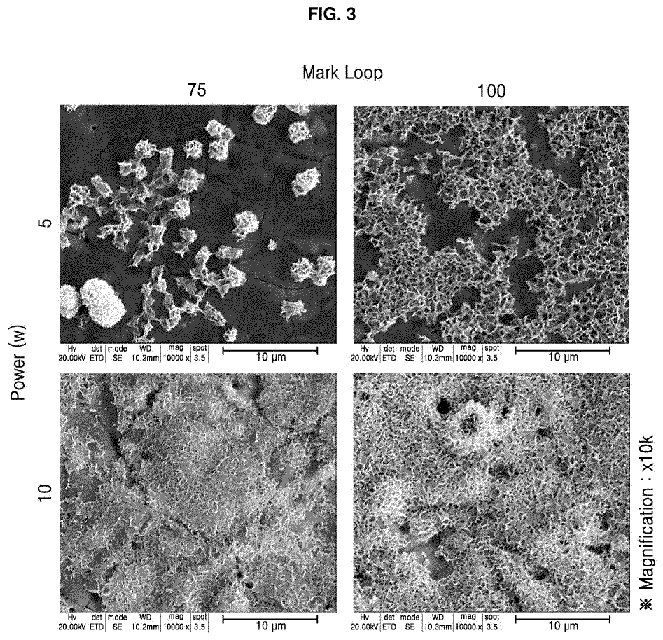

FIG. 3 shows SEM images of apatite formed according to the power of the laser beam and the repetition number of the laser beam emission onto the titanium alloy substrate. It may be confirmed that a larger amount of apatite is formed when the power was 10 W and the number of repetition was 100 (Example 4) than when the power was 5 W and the number of repetition was 75 (Example 1). That is, the amount of apatite formed on the surface of the substrate increases as the energy of the laser beam increases and the number of repetition increases.

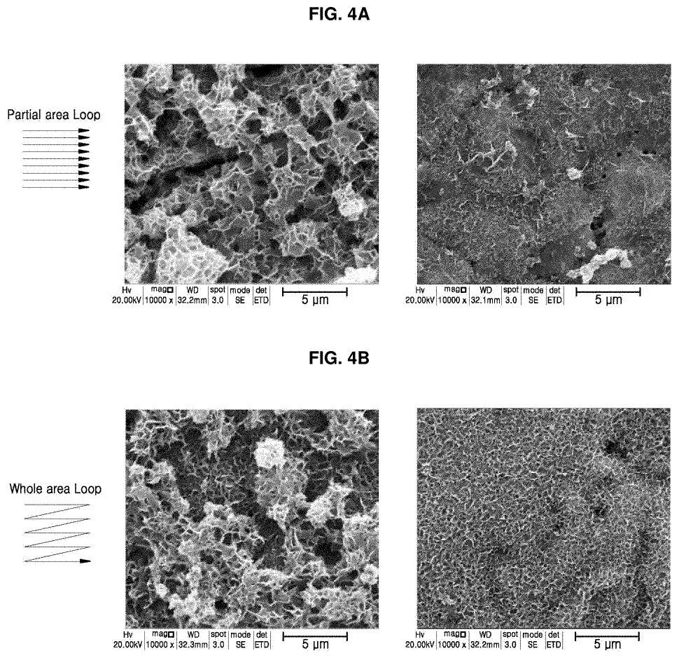

Differences of apatite formation according to the laser beam emission method were confirmed, and the results are shown in FIGS. 4A and 4B. Specifically, FIGS. 4A and 4B show SEM images of apatite formed on the surface of the substrate according to laser beam emission methods of Examples 5 and 6, respectively, and it may be confirmed that apatite was uniformly formed regardless of the laser beam emission method.

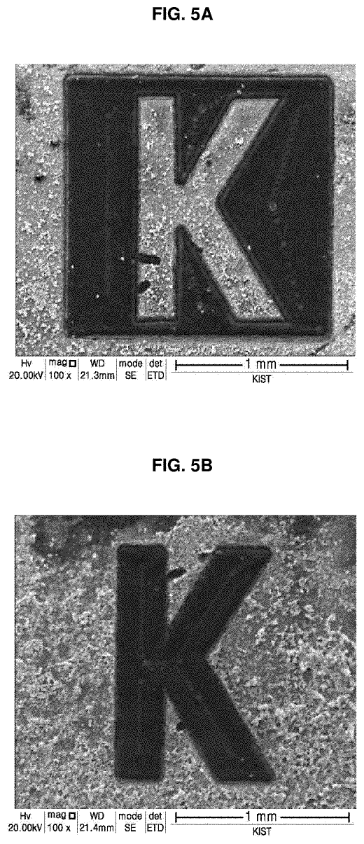

FIGS. 5A and 5B show cases in which apatite is formed on a partial region of the substrate.

FIG. 5A shows a result of forming a pattern in the English letter K on the substrate by repeating scanning of the substrate with the laser beam in the letter K shape 10 times under the same conditions as in Example 5 (where, a white portion indicates apatite).

Meanwhile, FIG. 5B shows a result obtained by a two-step process as follows. First, in a first step, apatite was formed over the entire surface of the substrate by repeating emission of the laser beam 10 times under the same conditions as in Example 5. Subsequently, in a second step, DMEM was removed from the precursor solution container, and the laser beam was directly emitted onto the surface on which apatite was previously formed under the same conditions to perform scanning with the laser beam in the shape of the letter K. During the second step, the apatite was removed by the high energy of the laser beam locally in the region to which the laser beam was applied. A black portion of FIG. 5B is the region from which apatite was removed.

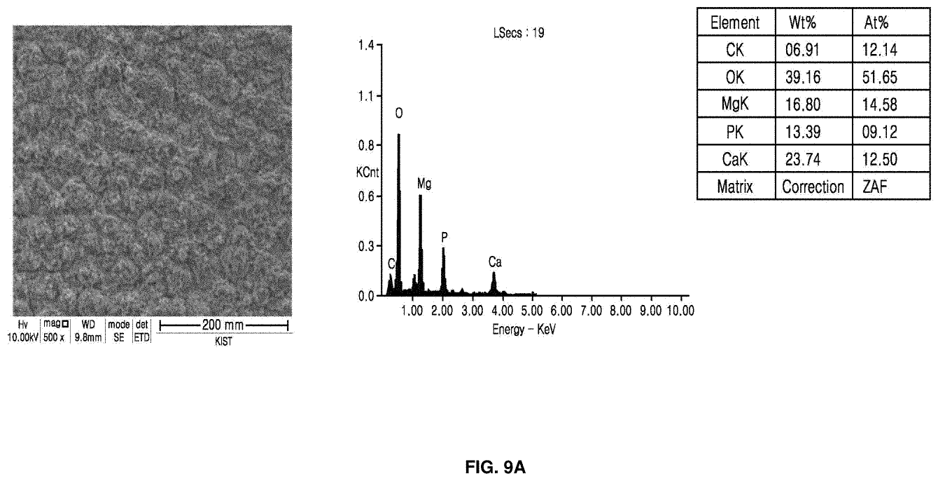

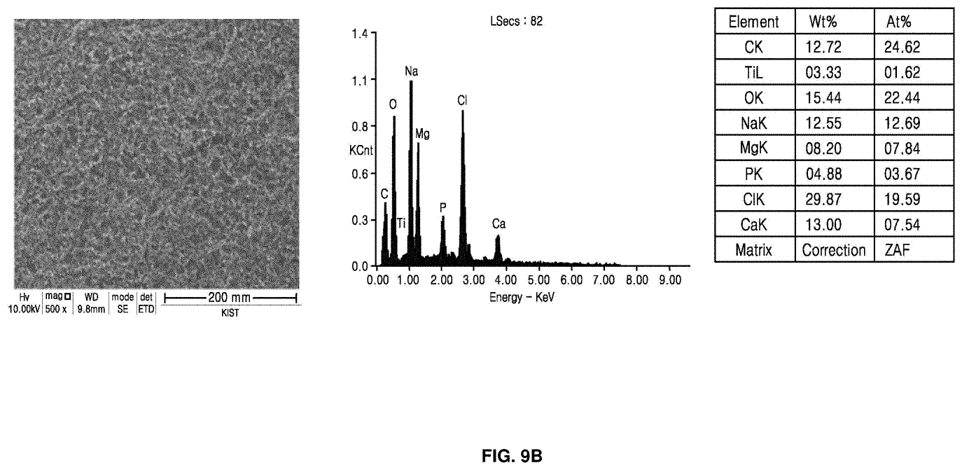

Based thereon, according to an embodiment of the present invention, it may be confirmed that either an embossed apatite pattern as shown in FIG. 9A or an engraved apatite pattern as shown in FIG. 9B may be formed on the surface of the substrate.

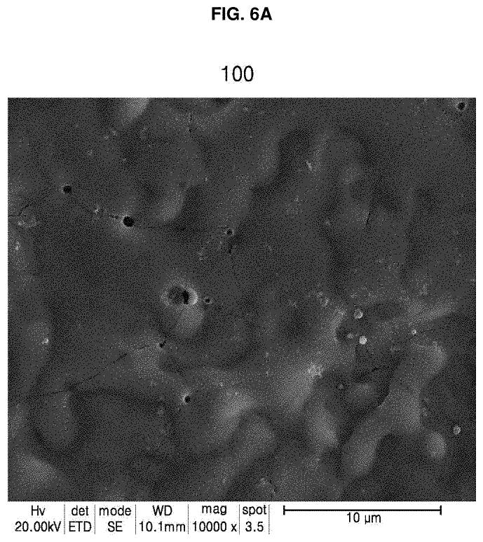

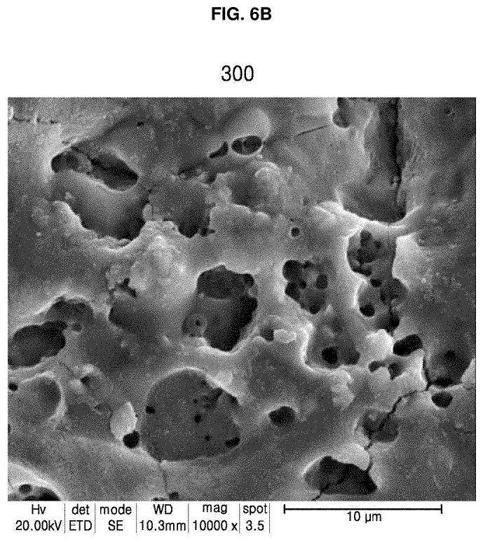

FIGS. 6A and 6B show SEM images indicating changes in surface morphology (roughness and pores) of titanium according to the repetition number of the laser beam emission according to an embodiment.

Referring to FIGS. 6A and 6B, it may be confirmed that pores were generated by repeated melting and solidification of the surface of titanium as the repetition number of of the laser beam emission increased.

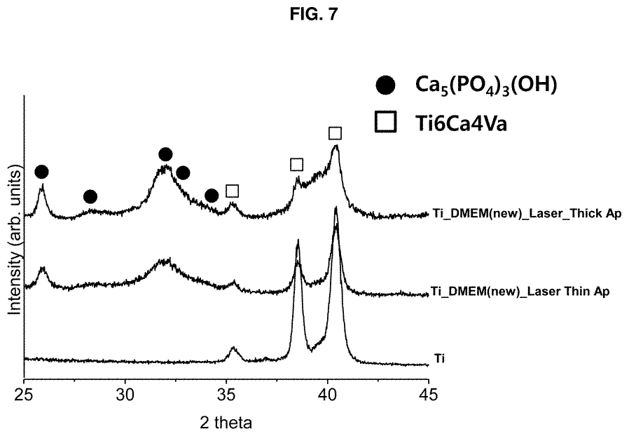

FIG. 7 shows X-ray diffraction (XRD) measurement results according to Examples 12 and 13, and it may be confirmed that X-ray diffraction peaks corresponding to Ca₅(PO₄)₃(OH) were identified in hydroxyapatite. Thus, as described above, it may also be confirmed that the surface layer formed on the titanium alloy substrate is formed of a hydroxyapatite phase.

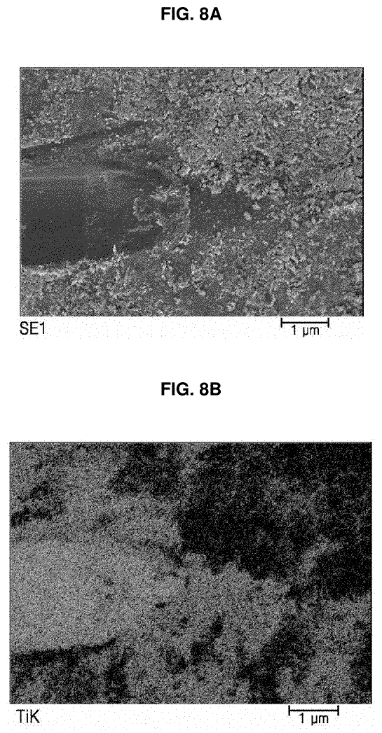

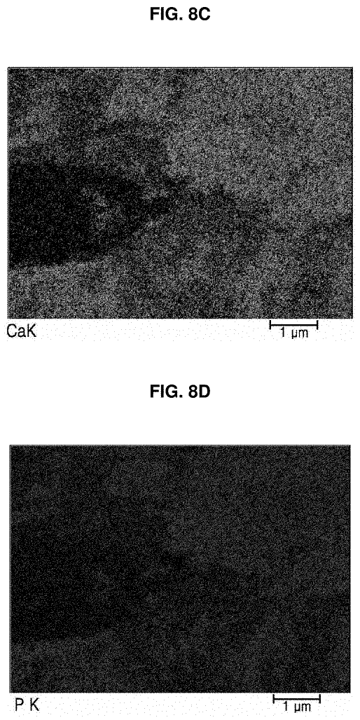

The apatite layers prepared according to Examples 12 and 13 were subjected to a scratching test to identify adhesion strengths thereof according to the thickness of the apatite coating layer, and the results are shown in Table 2. Residual depths shown in Table 2 are values obtained re-measuring portions where a probe penetrated. In addition, surface component analysis results by the scratching test are shown in FIGS. 8A to 8D. FIG. 8A is an SEM image of the surface after the scratching test, and FIGS. 8B to 8D show areas of Ti, Ca, and P components, respectively.

TABLE 2

| | Sample | Adhesion strength of | Thickness of coating |
|---|---|---|---|
| Example | No. | coating (N) | Residual depth (μm) |
| 12 | 7-1 | 31.9 | 10.2 |
| | 7-2 | 27.8 | 7.8 |
| | 7-3 | 35.3 | 7.7 |
| | average | 31.7 | 8.6 |
| 13 | 11-1 | 31.9 | 4.1 |
| | 11-2 | 75.7 | 11.7 |
| | 11-3 | 33.9 | 10.4 |
| | average | 47.2 | 8.7 |

First, referring to FIGS. 8A to 8D, it was confirmed that Ca and P were identified on the surface of the substrate even after the scratching test applied to the surface.

Thus, it may be confirmed that the apatite coating layer was not completely removed by the scratching test but remained on the surface of the substrate. These results indicate that the apatite coating formed according to the present embodiment has excellent adhesive force.

Referring to Table 2, it was confirmed that relatively thin apatite layers formed on the surfaces of the substrates according to Example 12 had an average adhesion strength of 31.7 N. It was also confirmed that relatively thick apatite layers formed on the surfaces of the substrates according to Example 13 had an average adhesion strength of 47.2 N or more.

The results of the examples were compared with adhesion strengths of apatite coatings formed according to conventional apatite coating methods, such as a plasma-spray method and a laser sputtering method, and the results are shown in Table 3.

TABLE 3

| | | | | Adhesion | |
|---|---|---|---|---|---|
| | | | Coating | strength | |
| | Substrate | Coating layer | process | (N) | Reference |
| 1 | titanium | hydroxyapatite | plasma-spray | 13.1 | J Biomed Mater |
| | | | method | | Res A. 2005 Mar |
| | | | | | 15; 72(4): 428-38. |
| 2 | titanium | hydroxyapatite | Laser | 0.0384 | J Mater Sci Mat Med |
| | | | sputtering | | 2002; 13: 253-258. |
| 3 | titanium | hydroxyapatite | Laser | 0.0017 | Appl Surf Sci |
| | | | deposition | | 2002; 195: 31-37 |
| | | | coating | | |
| 4 | titanium | hydroxyapatite | Laser | 9.6 | Biomaterials |
| | | | deposition | | 2003; 24: 3403-3408 |
| | | | coating | | |
| 5 | titanium | hydroxyapatite | Laser | 11.21 | J Mater Sci: Mater |
| | | | deposition | | Med (2011) 22: |
| | | | coating | | 1671. |

Referring to Table 3, while the hydroxyapatite coatings prepared according to the conventional methods exhibited a maximum adhesion strength of about 13.1 N, it was confirmed that the adhesion strength of the apatite coating obtained in Example 13 according to the present invention was a far higher value of 47.2 N.

Apatite was formed on a magnesium alloy substrate according to Examples 14 and 15, and analysis results thereof are shown in FIGS. 9A and 9B. FIG. 9A shows an SEM image of apatite and composition analysis results thereof by EDS according to Example 14 and FIG. 9B shows an SEM image of apatite and composition analysis results thereof by EDS according to Example 15.

Referring to FIGS. 9A and 9B, Ca and P peaks were identified as a result of analyzing the material formed on the surface of the magnesium alloy substrate by EDS.

Based thereon, it was confirmed that an apatite layer was formed on the surface of the magnesium alloy substrate. Thus, it may be confirmed that apatite is stably formed on the surface of the magnesium alloy substrate as well as on the titanium alloy substrate.

According to an embodiment of the present invention as described above, a method of forming an apatite coating by emitting a laser beam onto the surface of the substrate on which the precursor solution is applied may be provided.

However, these problems to be solved are illustrative and the scope of the present invention is not limited thereby.

While exemplary embodiments have been shown and described above, it will be apparent to those skilled in the art that modifications and variations could be made without departing from the scope of the present invention as defined by the appended claims.

What is claimed is:

1. An apparatus for forming an apatite coating comprising:
a precursor solution container to contain a precursor solution for forming apatite and providing an environment in which the precursor solution is in direct contact with a substrate; and
a laser generator disposed to emit a laser beam onto the substrate through the precursor solution contained in the precursor solution container in a state where the precursor solution is in direct contact with the substrate.

2. The apparatus of claim 1, further comprising a substrate receiving part on which the substrate is placed,
wherein the precursor solution container has an opening at one or more positions allowing the precursor solution contained therein to be in direct contact with the substrate.

3. The apparatus of claim 2, wherein the opening of the precursor solution container has a structure sealed by the substrate.

4. The apparatus of claim 1, further comprising a substrate receiving part on which the substrate is placed,
wherein the substrate receiving part is formed inside the precursor solution container.

5. A method of forming an apatite coating, the method comprising:
(a) brining an apatite-forming precursor solution comprising Ca²⁺ ions and PO₄³⁻ ions into direct contact with at least one region of the substrate;
(b) emitting a laser beam onto the region of the substrate in direct contact with the precursor solution through the precursor solution; and
(c) forming apatite in the region onto which the laser beam is emitted.

6. The method of claim 5, further comprising (d) partially removing the apatite by removing the precursor solution and emitting a laser beam onto the region on which the apatite is formed after the step (c).

7. The method of claim 5, wherein the precursor solution is selected from Dulbecco Modified Eagle Medium (DMEM), human blood plasma (HBP), and simulated body fluid (SBF).

8. The method of claim 5, wherein the precursor solution is concentrated to 1 to 400 times for use.

9. The method of claim 5, wherein the emitting of the laser beam is performed by repeatedly scanning the laser beam once or more times in one direction by a predetermined distance.

10. The method of claim 5, wherein the emitting of the laser beam is performed by repeatedly scanning the laser beam once or more times in a zigzag direction by a predetermined distance.

11. The method of claim 5, wherein the substrate comprises one of titanium (Ti), a titanium alloy, magnesium (Mg), and a magnesium alloy.
